(19) United States
(12) Patent Application Publication
(10) Pub. No.: US 2020/0234795 A1
(43) Pub. Date: Jul. 23, 2020

(54) Pruning Pair-HMM Algorithm And Hardware Architecture

(71) Applicant: THE REGENTS OF THE UNIVERSITY OF MICHIGAN, Ann Arbor, MI (US)

(72) Inventors: Xiao WU, Ann Arbor, MI (US); Satish NARAYANASAMY, Ann Arbor, MI (US); Reetuparna DAS, Ann Arbor, MI (US); David T. BLAAUW, Ann Arbor, MI (US)

(21) Appl. No.: 16/749,039

(22) Filed: Jan. 22, 2020

Related U.S. Application Data
(60) Provisional application No. 62/795,159, filed on Jan. 22, 2019.

(51) Int. Cl. G16B 30/10; G16B 20/20

(52) U.S. Cl. CPC G16B 30/10; G16B 20/20

(57) ABSTRACT

A method is presented for aligning a read with a haplotype. The method includes: constructing an overall matrix for computing alignment probabilities between a given read and a given haplotype, calculating, during a first pass, an alignment probability for each cell in the overall matrix using Pair-HMM method, where the alignment probabilities are calculated using fixed-point arithmetic; pruning cells from the overall matrix to derive a subset of unpruned cells; and calculating, during a second pass, an alignment probability for each cell in the subset of unpruned cells using the Pair-HMM method, where the alignment probabilities are calculated using floating-point arithmetic.

### CROSS-REFERENCE TO RELATED APPLICATIONS

This application claims the benefit of U.S. Provisional Application No. 62/795,159, filed on Jan. 22, 2019. The entire disclosure of the above application is incorporated herein by reference.

### FIELD

The present disclosure relates to an improved method for aligning a read with a haplotype during DNA sequencing.

### BACKGROUND

Recent advances in next-generation sequencing have enabled fast DNA sequencing for cancer, genetic disorder and pathogen detection. Short DNA fragments are sequenced in a massively paralleled method, producing billions of DNA reads (strings of ˜100 nucleotides: A, C, G, T) per human genome. Reassembling these DNA fragments to determine differences from a common reference genome (secondary analysis) requires extensive computation. Among several secondary analysis steps, variant calling is the final step which identifies disease related gene mutations and remains extremely time-consuming. In particular, the pair-Hidden-Markov-Model (Pair-HMM) forward algorithm (or PFA) requires ˜250T FLOPs for variant calling to infer mutation probabilities and contributes 70% of variant calling latency. Pair-HMM forward algorithm requires alignment matrix calculation, with a complicated combination of floating point addition and multiplication to infer overall similarity of two strings, making it a difficult hardware optimization problem. Pair-HMM forward algorithm has been mapped to a GPU as well as a systolic array and ring-based array on an FPGA. However, these methods are still constrained by the availability of floating point resources, with the hardware optimization mainly improving processor element (PE) utilization. Furthermore, no dedicated ASIC has been demonstrated to date to accelerate the Pair-HMM forward algorithm for DNA sequencing.

This section provides background information related to the present disclosure which is not necessarily prior art.

### SUMMARY

This section provides a general summary of the disclosure, and is not a comprehensive disclosure of its full scope or all of its features.

A method is presented for aligning a read with a haplotype. The method includes: constructing an overall matrix for computing alignment probabilities between a given read and a given haplotype, calculating, during a first pass, an alignment probability for each cell in the overall matrix using Pair-HMM method, where the alignment probabilities are calculated using fixed-point arithmetic; pruning cells from the overall matrix to derive a subset of unpruned cells; and calculating, during a second pass, an alignment probability for each cell in the subset of unpruned cells using the Pair-HMM method, where the alignment probabilities are calculated using floating-point arithmetic.

Pruning cells from the overall matrix further comprises identifying a dominant section of cells in the overall matrix and setting the alignment probability of remaining cells to zero, where the dominant section of cells represent an alignment between the read and the haplotype with highest probability. More specifically, pruning cells from the overall matrix may further include: a) identifying a seed cell in the overall matrix, where the seed cell is the cell having largest alignment probability in bottom row on the overall matrix; b) determining whether the alignment probability of the seed cell is dominate over an adjacent cell along a diagonal extending towards upper left of the overall matrix; c) setting the alignment probability of cells in same row as the seed cell to zero and setting the alignment probability of cells in same column as the seed cell to zero in response to a determination that the alignment probability of the seed cell is dominate over the adjacent cell; and d) repeating steps b) and c) for each adjacent cell along the diagonal extending from the seed cell until the alignment probability of a given cell along the diagonal is not dominate over the adjacent cell.

In some embodiments, at least one of the given read or the given haplotype is extracted from a biological sample.

In other embodiments, the method further includes selecting a mutation with highest likelihood based in part of the alignment probability for each cell in the subset of unpruned cells.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram illustrating variant calling;

FIG. 2 is a diagram illustrating the conventional Pair-HMM forward algorithm;

FIG. 3 is a flowchart illustrating an improved method for aligning a read with a haplotype;

FIGS. 4A and 4B are diagrams conceptually illustrating the pruning process;

FIGS. 5A-5C are diagrams depicting an example technique for pruning cells from the matrix;

FIG. 6 is a diagram depicting an alternative technique for pruning cells from the matrix;

FIG. 7 is a diagram of an example implementation of a hardware architecture for a pruning-based accelerator;

FIG. 8 is a diagram showing an example hardware implementation of a scan machine;

FIG. 9 is a diagram showing a circuit implementation for an integer processor element; and

FIGS. 10A and 10B are graphs comparing results of the prune-based accelerator to conventional approaches.

Corresponding reference numerals indicate corresponding parts throughout the several views of the drawings.

### DETAILED DESCRIPTION

Example embodiments will now be described more fully with reference to the accompanying drawings.

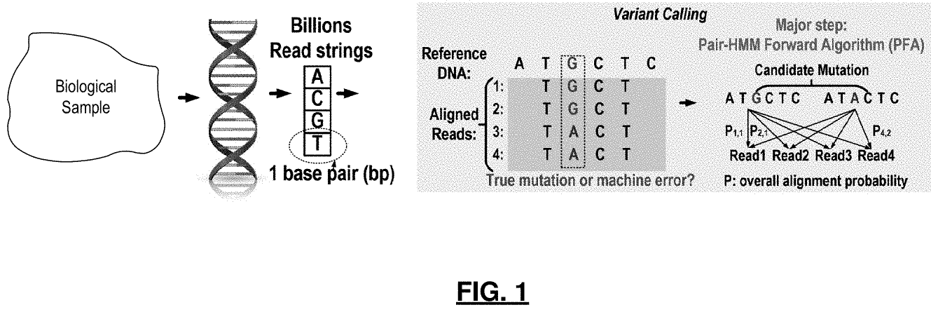

A genome is a long string of DNA base-pairs A, G, C, and T. Sequencers produce chopped and out-of-order reads from biological samples which are then reconstructed to form sequence whole genome. To further identify mutations of a person's genome, aligned reads need to be compared against a reference genome. This process is referred to as variant calling as seen in FIG. 1. Among the steps in variant calling, aligning a read with a haplotype using the Pair-HMM method accounts for 70% computation time.

This disclosure present a pruning-based Pair-HMM algorithm and its ASIC implementation for high throughput DNA variant calling. The algorithm-architecture co-design identifies high-quality alignment regions in input data, and devotes floating point resources only to these regions, thereby aggressively reducing expensive floating point computation. For non-high-quality regions, floating point multiplication is replaced with low accuracy 20b integer addition by using log domain number representation, while maintaining (provable) correctness in the downstream analysis. Floating point computation is reduced by 43× on real human genome data, and is replaced by low accuracy integer PEs (I-PEs) that are 4.6× smaller in area and 1.9× higher in performance than floating point PEs (FP-PEs). Implemented in 40 nm CMOS, the 5.67 mm2 accelerator demonstrates 17.3G cell updates per second (CUPS) throughput, marking a 6.6 improvement over a baseline ASIC implementation with the conventional floating-point-only algorithm.

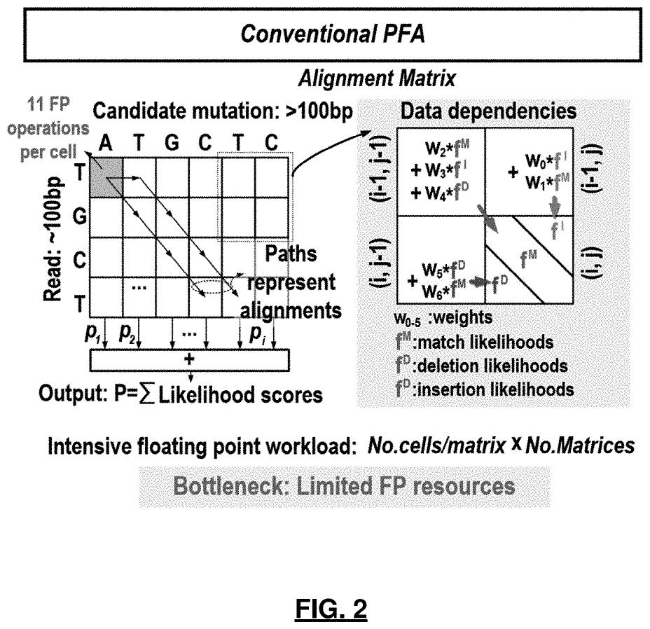

Pair-HMM is a statistical model which determines per-read likelihoods of each haplotypes of the individual. It helps determine the real DNA expression of an individual given the possibly incorrect reads. Conventional pair-HMM forward algorithm calculates probabilities of all alignments between a candidate mutation string and a DNA read using an alignment matrix as seen in FIG. 2. The likelihood of a particular cell (i,j), representing a particular alignment, is compute from the likelihood of its three neighboring cells: vertical neighbor cell (i−1,j) representing an insert transition, diagonal neighbor (i−1,j−1) for a match transition, and horizontal neighbor (i,j−1) for a delete transitions. A key observation is that the final score of the matrix is typically dominated by the probabilities of only a few alignment paths, thanks to high quality reads and a small likelihood of genetic mutations. While reference is made herein to Pair-HMM, other techniques for predicting or determining sequence alignments also fall within the broader aspects of this disclosure.

Forward algorithm is commonly used in Pair-HMM model, which efficiently calculates the overall probability of all possible alignments between read and haplotype. The forward algorithm is essentially a probability based dynamic programming approach, which uses three matrices f^{M}, f^{I}, f^{D}. f^{k}(i,j) corresponds to the combined probability of all alignments up to position (i,j) of read and haplotype that ends in state k. k can be I (insertion), D (deletion) and M (match). For each position (i,j), f^{M}, f^{I}, f^{D} are calculated as below:

f^{M}(i,j)=pₘₘf^{M}(i−1,j−1)+pᵢₘf^{I}(i−1,j−1)+p_{dm}f^{D}(i−1,j−1) (1)

f^{I}(i,j)=aₘᵢf^{M}(i−1,j)+aᵢᵢf^{I}(i−1,j) (2)

f^{D}(i,j)=a_{md}f^{M}(i,j−1)+a_{dd}f^{D}(i,j−1) (3)

where pₘₘ, p_{dm}, aₘᵢ, aᵢᵢ, a_{md}, and a_{dd} are probabilities related to state transition and read quality score. Final output of forward algorithm is sum of insertion and match probabilities in the final row: Σⱼ₌₁^{L}^{ₕ}(Lᵣ,j)+f^{I}(Lᵣ,j), where Lᵣ and Lₕ are the length of read and haplotype. As can be seen above, the forward algorithm is based on probabilities which can get very small quickly and thus requires computational intensive floating point calculation.

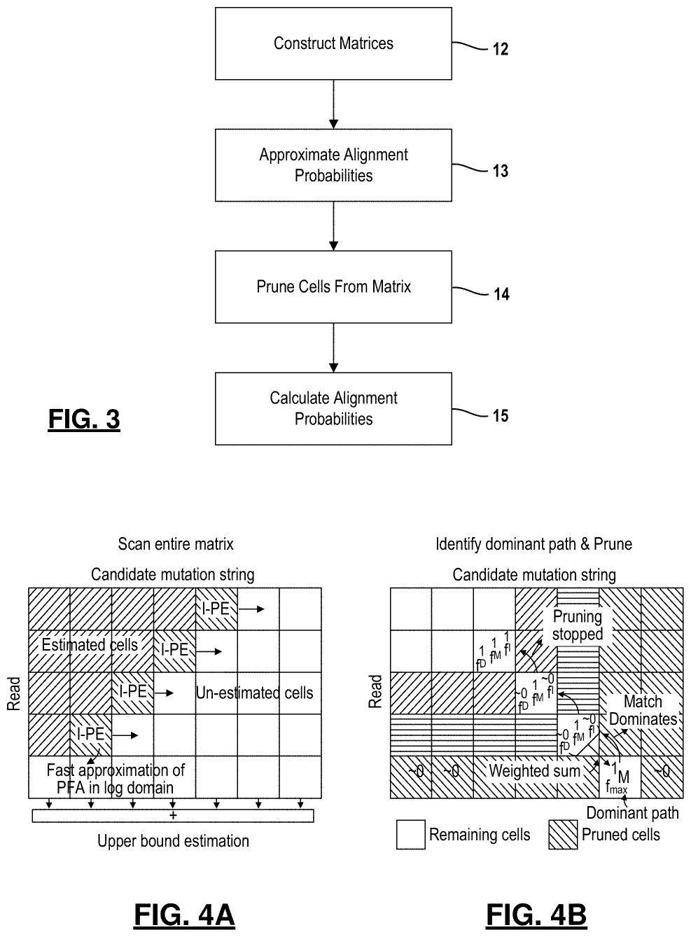

FIG. 3 illustrates an improved method 10 for aligning a read with a haplotype. In the Pair-HMM model, three matrices are constructed at 12 for computing alignment probabilities between a given read and a given haplotype: one for match state, one for insertions state and one for deletion state. For each matrix, each row in the matrix corresponds to a character in the given read, each column in the matrix corresponds to a character in the given haplotype, and each cell in the matrix represents an alignment probability between characters of the given read and the given haplotype.

During a first pass (or scan phase), an alignment probability for each cell in each of the three matrices is calculated or approximated at 13 using the Pair-HMM method. Of note, the alignment probabilities are calculated using the less computationally intensive fixed-point arithmetic. That is, an upper bound likelihood for each cell in the matrix is computed using integer processor elements operating in logarithmic number representation which replaces multiplication with addition and significantly reduces hardware complexity. Other approximation techniques for computing alignment probabilities are also contemplated by this disclosure.

In order to calculate overall alignment probability of read-haplotype pair, the Pair-HMM method calls for summing f^{M}, f^{I} and f^{D} from adjacent cells at each position (i,j). FIG. 2 illustrates data dependencies for f^{M}(i,j), f^{I}(i,j) and f^{D}(i,j) according to equation (1-3). For example, f^{M} in each square (indexed (i,j)) depends on weighted sum of f^{M} f^{I} and f^{D} from the square before it along the diagonal line (indexed (i−1,j−1)). In many cases, a key observation is that weighted f^{I}(i−1,j−1) and f^{D}(i−1,j−1) are much smaller than f^{M}(i−1,j−1). This means that setting f^{I}(i−1,j−1) and f^{D}(i−1,j−1) to zero (i.e. prune them) leads to negligible loss in the result f^{M}(i,j). As one continues to calculate the overall matrix, if f^{I}(i,j) and f^{D}(i,j) are significantly smaller compare to f^{M}(i,j), one can prune f^{I}(i,j) and f^{D}(i,j) without sacrificing the accuracy of f^{M}(i+1,j+1). Based on this observation, cells can be pruned from the overall matrix as indicated at 14 of FIG. 1.

Conceptually, pruning cells from the overall matrix is achieved by identifying a dominant section of cells in the overall matrix and setting the alignment probability of remaining cells to zero as shown in FIGS. 4A and 4B, where the dominant section of cells represent an alignment between the read and the haplotype with highest probability. The dominant section of cells is also referred to herein as a subset on unpruned cells from the matrix.

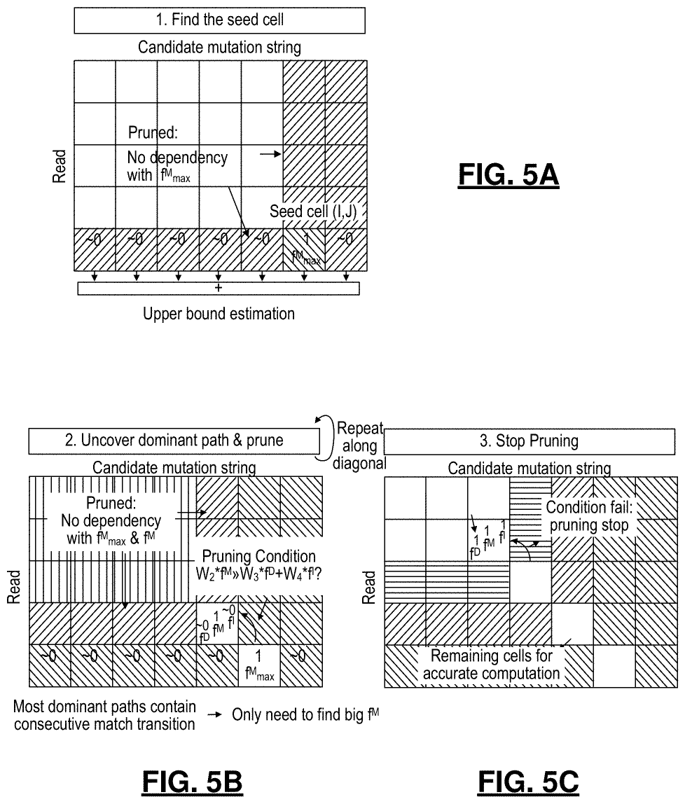

FIG. 5A-5C further illustrate one example technique for pruning cells from the overall matrix. First, a seed cell 51 is identified in the overall matrix as seen in FIG. 5A. The seed cell is identified as the cell having largest alignment probability in the bottom row on the overall matrix. In other words, the cell (I, J) with highest match score in the bottom row indicates the end position of a good alignment and is picked as seed position for pruning. Other techniques for identifying the seed cell are also contemplated by this disclosure.

Next, a determination is made as to whether the alignment probability of the seed cell is dominate over an adjacent cell 52, where the adjacent cell is adjacent to the seed cell along a diagonal extending from the seed cell and towards an upper left of the overall matrix. In the case that the alignment probability of the seed cell is dominate over the adjacent cell, the alignment probability of cells in same row as the seed cell are set to zero and the alignment probability of cells in same column as the seed cell are set to zero as shown in FIG. 5B. In one embodiment, dominate means the alignment probability of seed cell is 10× or more larger that the alignment probability of the adjacent cell although in other embodiments the cutoff for dominate may be set up to two times or more.

For each adjacent cell in the diagonal extending from the seed cell, these steps of comparing alignment probabilities for diagonal adjacent cells and pruning cells is repeated until the alignment probability of the given cell in the diagonal is not dominate over the adjacent cell. In some instances, the pruning process may result in a single diagonal stretching from the bottom row of the matrix to the top row of the matrix and the cells comprising the diagonal are the subset of unpruned cells. In other instances, a stopping condition is reached before the diagonal reaches the top row as shown in FIG. 5C. That is, a given cell along the diagonal is not dominate over the adjacent cell. In these instances, the subset of unpruned cells includes the cells above the given cell in the overall matrix and the cells to left of the given cell in the overall matrix as well as the cells in the diagonal extended from the seed cell.

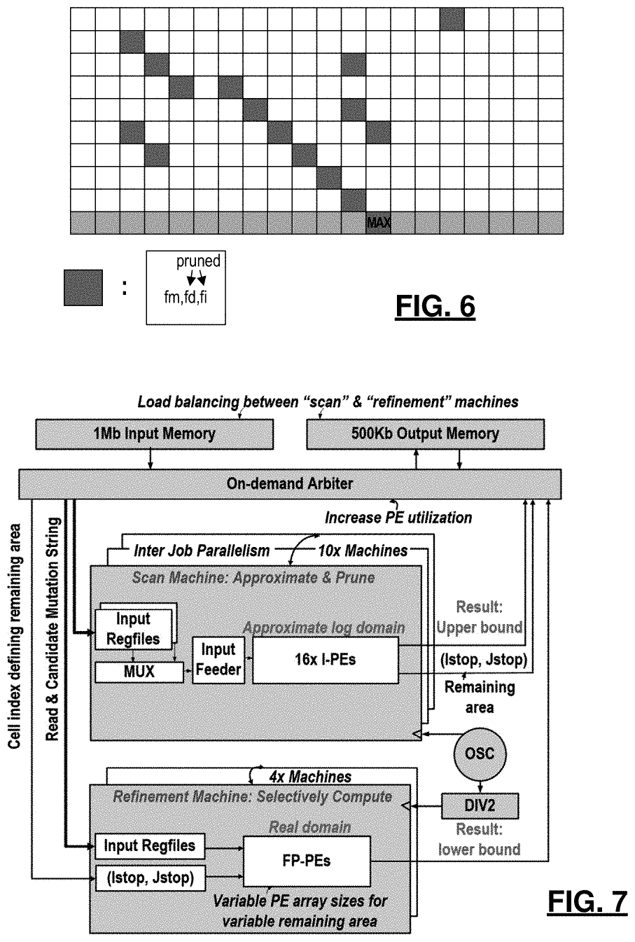

The pruning method described above starts with final row of the matrix and proceed backwards in the diagonal direction. This requires hardware to store the values of the entire matrix. FIG. 6 shows an alternative way of pruning without storing the entire matrix. During the scan phase, search for diagonals where all f^{M} along the diagonal is significantly larger than f^{I} and f^{D}, as one of these short diagonals may become the dominant diagonal described above. Only diagonals that are long enough to reach the final row are recorded. At the end of scan phase, a set of candidate diagonals is produced. Each of them starts in some cell in the middle of the matrix and ends in the final row. Then, search for cell (I,J) in the final row with the highest f^{M}. If the cell (I,J) happens to be the tail cell of one of the candidate diagonals, this diagonal is picked as the dominant diagonal. Its starting position will become (Istop, Jstop), which defines the unpruned region for refinement phase. Other pruning methods are also envisioned by this disclosure.

Returning to FIG. 3, an alignment probability is computed for each cell in the subset of unpruned cell during a second pass (or refinement phase) as indicated at 15. In the example embodiment, the alignment probabilities are computed using floating-point arithmetic.

Variant calling algorithm emits the final called variant by selecting the mutation with the highest likelihood from amongst all candidate mutations. The mutation likelihood is proportional to product of all per-read likelihoods (i.e. output of Pair-HMM). The proposed pruning hardware accelerator guarantees the correctness of final called variants by forcing the lower bound of called variant to have a higher probability of the upper of un-called variants. Upper bound of un-called variant is readily available from the pruning machine output. Lower bound of called variant is the result of floating point machine. Therefore one can achieve significant speedup and still guarantee the correct final result. The mutation with the highest likelihood is correctly determined as the final variant calling result in 98.5% of all cases. The failing cases (˜1.5%) are guaranteed to be identified by recomouting using only floating point processor elements.

To effectively implement the proposed prune based pair-hmm algorithm, an example implementation for a hardware architecture shown in FIG. 7. The accelerator consists of 10 scan machines composed of 16 integer processor elements (I-Pes) each to upper bound and prune matrices, and 4 refinement machines composed of floating point processor elements (FP-Pes) to accurately compute un-pruned regions. Refinement machines come in two sizes with 1× and 4×FP-PEs to accommodate the variable size of un-pruned regions. An on-demand arbiter streams in jobs from input memory, dispatches them to scan and refinement processor elements and streams results to output memory.

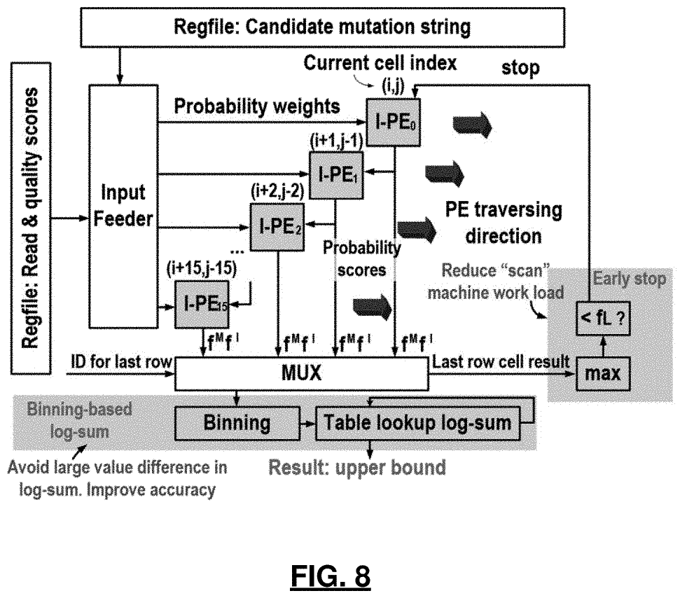

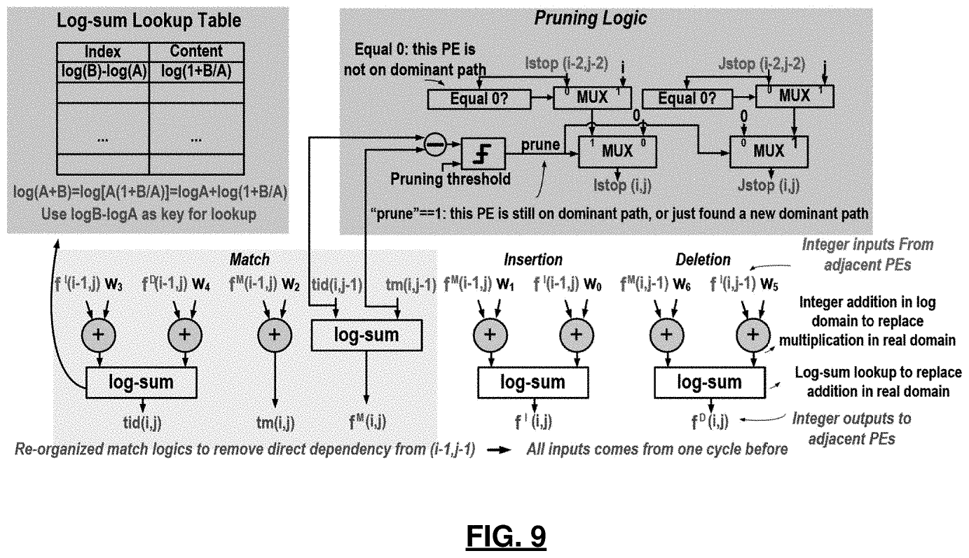

FIG. 8 shows an example hardware implementation of a scan machine, consisting of 16 processor elements, an input feeder to control processor element traversal across the matrix, a binning based log-sum module to avoid accuracy degradation in the last row, and an early stop detection module. Each processor element uses 20 bits fixed point addition and a 15-entry table lookup in log domain as substitutes for multiplication and addition in real domain respectively. Instead of tracing back to determine the pruned region, logic in processor elements prune cells as processor elements traverse forward across the matrix, avoiding the need to store scores for the entire matrix. Processor elements work in parallel when traversing the matrix from left to right. As processor elements traverse, an early detection module opportunistically stops the scan phase once the maximum score in one row is smaller than a threshold. This optimization takes advantage of downstream processing where extremely low Pair-HMM results are filtered completely, reducing workload even in the scan phase (by 18%). Because only adjacent processor elements communicate with each other, routing complexity is greatly reduced. An example circuit implementation of the I-PE is shown in FIG. 9.

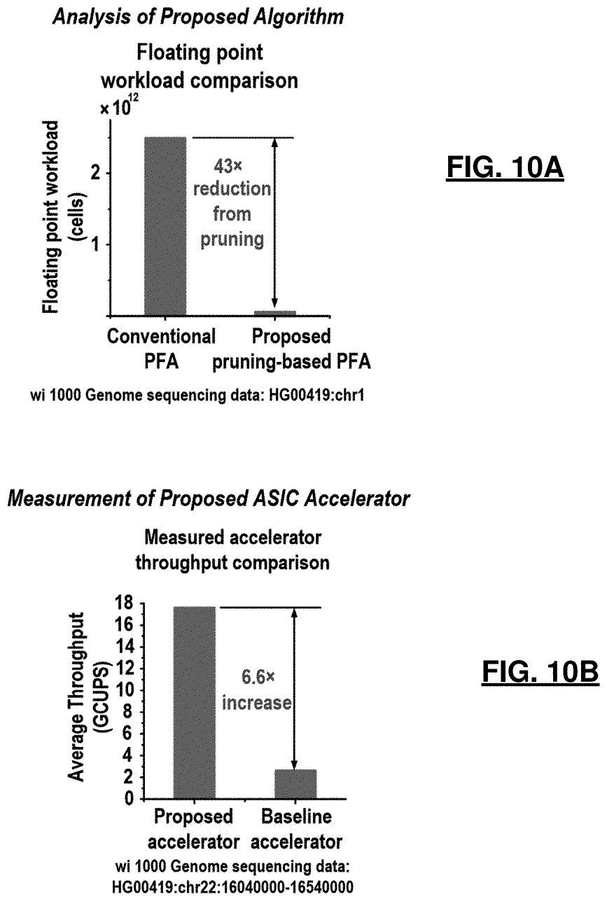

In an example embodiment, fabricated in 40 nm CMOS with 5.67 mm2 die area, the pruning-based accelerator runs at 120 MHz with 756 mW power consumption. FIGS. 10A and 10B show that the number of cells requiring floating point calculation is reduced by 43× (includes re-computation due to bound check failure). Executing only with the 4 FP-PE refinement machines (Fmax=62.5 MHz), one can determine the baseline ASIC performance of the conventional PFA algorithm (no pruning). Proposed accelerator was verified with real sequencing data and shows 17.3 GCUPS average throughput which is a 6.6× improvement over baseline ASIC implementation (normalized to same area). Speedups of 355× and 661× in CUPS/mm2 were obtained as compared to FPGA and NVidia K4 GPU implementations, respectively.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

In sum, the proposed algorithm includes a fast pruning machine implemented using fixed point calculation and an accurate machine which only works on unpruned cells using floating point calculation. In step one, the fast pruning machine calculates entire alignment matrix rapidly and approximately in log domain. The calculation can be done using approximation, including fixed point calculation with fewer bits to optimize speed. Log-sum is substituted with fast table lookup. Based on approximate values, the accelerator prunes squares in the matrix whose values contribute insignificantly to overall probabilities using the method introduced previously. In step two, precise machine using floating point representation calculates alignment probabilities on only the remaining squares, resulting a final probability slightly lower than accurate overall probability. Based on the proposed pruning technique, one can save 95% floating point computation, leading to a potential 20× reduction in execution time.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for aligning a read with a haplotype, comprising:
constructing an overall matrix for computing alignment probabilities between a given read and a given haplotype, where each row in the overall matrix corresponds to a character in the given read, each column in the overall matrix corresponds to a character in the given haplotype, and each cell in the overall matrix represents an alignment probability between characters of the given read and the given haplotype;
calculating, during a first pass, an alignment probability for each cell in the overall matrix using Pair-HMM method, where the alignment probabilities are calculated using fixed-point arithmetic;
pruning cells from the overall matrix to derive a subset of unpruned cells; and
calculating, during a second pass, an alignment probability for each cell in the subset of unpruned cells using the Pair-HMM method, where the alignment probabilities are calculated using floating-point arithmetic.

2. The method of claim 1 further comprises calculating an alignment probability for each cell as an order of magnitude in log domain during the first pass.

3. The method of claim 1 wherein pruning cells from the overall matrix further comprises identifying a dominant section of cells in the overall matrix and setting the alignment probability of remaining cells to zero, where the dominant section of cells represent an alignment between the read and the haplotype with highest probability.

4. The method of claim 1 wherein pruning cells from the overall matrix further comprises
a) identifying a seed cell in the overall matrix, where the seed cell is the cell having largest alignment probability in bottom row on the overall matrix;
b) determining whether the alignment probability of the seed cell is dominate over an adjacent cell along a diagonal extending towards upper left of the overall matrix;
c) setting the alignment probability of cells in same row as the seed cell to zero and setting the alignment probability of cells in same column as the seed cell to zero in response to a determination that the alignment probability of the seed cell is dominate over the adjacent cell;
d) repeating steps b) and c) for each adjacent cell along the diagonal extending from the seed cell until the alignment probability of a given cell along the diagonal is not dominate over the adjacent cell.

5. The method of claim 4 further comprises adding cells above the given cell in the overall matrix to the subset of unpruned cells and adding cells to left of the given cell in the overall matrix to the subset of unpruned cells.

6. The method of claim 4 wherein constructing an overall matrix further comprises constructing three matrices for computing alignment probabilities between the given read and the given haplotype and combining alignment probabilities from the three matrices to form the overall matrix, where cells in a first matrix represent alignment probability between characters of the given read and the given haplotype that ends with a match state, cells in a second matrix represent alignment probability between characters of the given read and the given haplotype that ends with a insertion, and cells in a third matrix represent alignment probability between characters of the given read and the given haplotype that ends with a deletion.

7. The method of claim 6 further comprises determining whether the alignment probability of the seed cell is dominate over an adjacent cell by comparing product of the alignment probability between characters of the given read and the given haplotype that ends with a match state for the seed cell and a weight for the seed cell with sum of a first product and a second product, where the first product is product of the alignment probability alignment probability between characters of the given read and the given haplotype that ends with a insertion for the adjacent cell and an insertion weight, and the second product is product of alignment probability between characters of the given read and the given haplotype that ends with a deletion for the adjacent cell and a deletion weight.

8. The method of claim 5 wherein combining alignment probabilities from the three matrices further comprises comparing f^{I}(i,j) to f^{M}(i,j) and comparing f^{D}(i,j) to f^{M}(i,j), and setting f^{k}(i,j) equal to f^{M}(i,j) when f^{I}(i,j) and f^{D}(i,j) are significantly smaller than f^{M}(i,j), where f^{k}(i,j) is alignment probability in the overall matrix.

9. The method of claim 1 further comprises extracting at least one of the given read and the given haplotype from a biological sample.

10. The method of claim 1 further comprises selecting mutation with highest likelihood based in part of the alignment probability for each cell in the subset of unpruned cells.

11. A method for aligning a read with a haplotype, comprising:
extracting a given read from a biological sample;
constructing an overall matrix for computing alignment probabilities between the given read and a given haplotype, where each row in the overall matrix corresponds to a character in the given read, each column in the overall matrix corresponds to a character in the given haplotype, and each cell in the overall matrix represents an alignment probability between characters of the given read and the given haplotype;
calculating an alignment probability for each cell in the overall matrix using Pair-HMM method, where the alignment probabilities are calculated using fixed-point arithmetic;
pruning cells from the overall matrix by identifying a dominant section of cells in the overall matrix and setting alignment probability for the remaining cells to zero, where the dominant section of cells represent an alignment between the read and the haplotype with highest probability;
calculating an alignment probability only for cells in the dominant section of cells using the Pair-HMM method, where the alignment probabilities are calculated using floating-point arithmetic.

12. The method of claim 11 further comprises calculating an alignment probability for each cell in the overall matrix as an order of magnitude in log domain.

13. The method of claim 11 wherein pruning cells from the overall matrix further comprises
e) identifying a seed cell in the overall matrix, where the seed cell is the cell having largest alignment probability in bottom row on the overall matrix;
f) determining whether the alignment probability of the seed cell is dominate over an adjacent cell, where the adjacent cell is adjacent to the seed cell along a diagonal extending from the seed cell and towards an upper left of the overall matrix;
g) setting the alignment probability of cells in same row as the seed cell to zero and setting the alignment probability of cells in same column as the seed cell to zero in response to a determination that the alignment probability of the seed cell is dominate over the adjacent cell;
h) for each adjacent cell in the diagonal extending from the seed cell, repeating steps b) and c) for a given cell in the diagonal until the alignment probability of the given cell in the diagonal is not dominate over the adjacent cell.

14. The method of claim 13 further comprises adding cells above the given cell in the overall matrix to the dominant section of cells and adding cells to left of the given cell in the overall matrix to the dominant section of cells.

15. The method of claim 14 wherein constructing an overall matrix further comprises constructing three matrices for computing alignment probabilities between the given read and the given haplotype and combining alignment probabilities from the three matrices to form the overall matrix, where cells in a first matrix represent alignment probability between characters of the given read and the given haplotype that ends with a match state, cells in a second matrix represent alignment probability between characters of the given read and the given haplotype that ends with a insertion, and cells in a third matrix represent alignment probability between characters of the given read and the given haplotype that ends with a deletion.

16. The method of claim 15 further comprises determining whether the alignment probability of the seed cell is dominate over an adjacent cell by comparing product of the alignment probability between characters of the given read and the given haplotype that ends with a match state for the seed cell and a weight for the seed cell with sum of a first product and a second product, where the first product is product of the alignment probability alignment probability between characters of the given read and the given haplotype that ends with a insertion for the adjacent cell and an insertion weight, and the second product is product of alignment probability between characters of the given read and the given haplotype that ends with a deletion for the adjacent cell and a deletion weight.
